(19) United States
(12) Patent Application Publication
(10) Pub. No.: US 2012/0157767 A1
(43) Pub. Date: Jun. 21, 2012

(54) Digital Cerviscopy Device and Applications

(75) Inventor: MONCEF JENDOUBI, SAN FRANCISCO, CA (US)

(73) Assignee: MILAGEN, INC., EMERYVILLE, CA (US)

(21) Appl. No.: 12/973,268

(22) Filed: Dec. 20, 2010

(51) Int. Cl. A61B 1/04; H04N 7/18

(52) U.S. Cl. 600/109; 348/68; 348/E07.085

(57) ABSTRACT

The present invention relates to a digital system for cervical disease screening and detection, with clinical application as adjunct to Pap smear. The device of the present invention is designed for insertion into a woman's genital cavity, and is composed of a digital camera and light source at one end to take pictures of the cervix and its environment, of a flexible tubing connecting said end to a distal end, and of a distal end comprising a digital mean with capacity to store images, display, transfer and share them as required, on-site or at a remote location. This instrument can either be used by a health professional, or self-operated.

The present invention provides a system making cervical disease screening routinely accessible to every woman, by improving frequency and cost-effectiveness of cervical disease surveillance in general, and facilitating access to health care professionals even to women in rural or underserved areas.

### FIELD OF THE INVENTION

The field of the invention relates to cervical disease detection, screening and management, using an easy-to-use flexible digital device capable of taking images and/or video images of the cervix and vaginal cavity, storing, and displaying them via digital means.

### BACKGROUND

Incidence and mortality. In the US in 2010 there were an estimated 12,200 new cases of invasive CvC and an estimated 4,210 deaths from CvC (American Cancer Society, ACS Cancer Facts & Figures, 2010; Jemal A, et al., Cancer statistics, CA Cancer J Clin 60:277-300, 2010). Since its establishment in 1957, Pap smear has become a routine screening test in the US, thus reducing CvC mortality (Kavita et al., Accuracy of the Papanicolaou test in screening for and follow-up of cervical cytologic abnormalities: a systematic review, Ann Int Med 132(10):810-819, 2000).

In the US each year approximately 50 million women undergo cytological screening (Wright T C Jr., Cox J T, Massad L S, Twiggs L B and Wilkinson E J, Consensus Guidelines for the management of women with cervical cytological abnormalities, Jama 287(16): 2120-2129, 2001), with some 7% (3.5 million) requiring additional follow-up (Jones B A and Davey D D, Quality management in gynecologic cytology using interlaboratory comparison, Arch Pathol Lab Med 124(5):672-81, 2000). It is estimated that the cost for colposcopic follow up and interventional treatment of abnormal cytological screening approaches 6 billion dollars annually in the US (Kurman R J, Henson D E, Herbst A L, Noller K L and Schiffman M H, Interim guidelines for management of abnormal cervical cytology, The 1992 National Cancer Institute Workshop, Jama 271(23): 1866-1869, 1994).

However, many countries lack the medical infrastructure or technical expertise to carry out effective CvC mass screening (Michelow et al., Simulation of primary cervical cancer screening by the PAPNET system in an unscreened, high-risk community, Acta Cytol 41(1):88-92, 1997; Veneti, et al., PAPNET for cervical cytology screening: experience in Greece. Acta Cytol 43(1):30-33, 1999; Denny et al., Two-stage cervical cancer screening: an alternative for resource-poor settings, Am J Obst Gyn 183 (2):383-388, 2000). As a result, CvC remains a major problem in many countries in the world and it is the leading cancer-related cause of death in women in the developing world. CvC is the second most commonly diagnosed malignancy and the third leading cause of cancer death in women worldwide. There were 555,100 new cases and 309,800 deaths estimated in 2007, 83% of which occurred in the developing world (American Cancer Society, ACS Global Cancer Facts and Figures, 2007).

Cervical cancer etiology and pathology. Infection with human papillomavirus (HPV) is a primary etiologic factor in CvC. Among the 200 HPV types known, HPV16/18 are the most commonly associated to CvC, with more than a 200-fold increased risk (Castellsague X, et al., Worldwide human papillomavirus etiology of cervical adenocarcinoma and its cofactors: implications for screening and prevention, J Natl Cancer Inst 5:303-315, 2006). However most HPV infections disappear spontaneously and only a small percentage progress to CIN (cervical intraepithelial neoplasia) or CIS (carcinoma in situ).

In general, CvC progresses slowly through several well-defined stages, and thus early detection permits the cancerous lesions to be treated with nearly 100% success. CvC comprises two major types: squamous cell carcinoma (75%) and adenocarcinoma (20%), affecting the squamous cells and the glandular cells of the cervix epithelium respectively (WHO/ICO, Human Papillomavirus and Cervical Cancer, Summary Report, Brazil, 2007; ACS, 2010).

CvC starts as a low grade squamous intraepithelial lesions (LSIL) or cervical intraepithelial neoplasia (CIN I), characterized by mild dysplasia. If not treated, this stage evolves into high grade squamous intraepithelial lesions (HSIL) or cervical intraepithelial neoplasia, including carcinoma in situ (CIN II, CIN III/CIS) characterized by moderate to severe dysplasia. CIN II carries a risk of progression into cancer of 16% by two years, and 25% after five years, if left untreated. CIS is cervix confined cancer that will develop into invasive cervical cancer (ICC) over a period of 10 to 12 years. One year and 5-year relative survival for CvC patients in the US is 88% and 72% respectively, while the 5-year survival rate for patients diagnosed with localized CvC is 92% (ACS, 2010). FIGS. 3 and 4 illustrate cervical cancer development.

Cervical cancer screening. The standard method of CvC screening uses the Pap smear, named after Dr. George Papanicolaou, who introduced it into clinical practice in the 1930s. Pap smear screening is presently a two-step process of collection and inspection. First, a medical professional collects a sample of cells from the cervix of the patient using a spatula, a brush, or cotton swab. Cells are directly smeared on a slide, fixed, and stained, and slides are read by a cytopathologist.

According to the ACS recommendations (Smith R A, Cokkinides V, Eyre H J. American Cancer Society Guidelines for the Early Detection of Cancer, CA Cancer J Clin 55:31-44, 2005). CvC screening should be done every year with conventional or LBC Pap test. If Pap smear is abnormal and reveals ASCUS, HPV testing is done, and if positive, women are referred to colposcopy. If Pap smear reveals LSIL or HSIL, women are immediately referred to colposcopy. Colposcopy is the microscopic examination of the cervix upon acetic acid or Lugol's stain to reveal abnormal cells, which can be in turn biopsied. Women over age 30 who have had three normal Pap test results in a row may get screened every 2-3 years with cervical cytology alone, or every 3 years with an HPV DNA test plus cervical cytology. Women over age 70 and older who have had three or more normal Pap test in a row, and no abnormal Pap test in the last 10 years, may stop screening (Smith, 2005).

Limitations of Pap smear screening. There are inherent limitations to the standard Pap smear screening related to the quality of the sample, the quality of the slide, and the effectiveness of the screener. The standard technique of placing the sampling spatula or brush on the glass slide results in capturing only the cells that are in contact with the slide. There is generally no proportional representation on the slide of all the cells taken from the cervix. In some cases, an inadequate number of cells are preserved on the slide, resulting in the need for re-screening. Even when the number of cells is adequate, the appearance of the resultant slide can be highly variable. The cells may be clumped, overlapping, and poorly preserved. Visibility may be partially obscured by blood, inflammation or drying artifacts.

There are also several factors that limit the effectiveness of the slide screener. First, a typical Pap smear slide contains up to 300,000 cells. With a limited amount of time to screen each slide, the screener cannot examine each cell, but instead must do a quick overall scan of the slide and then sample the most promising areas at greater magnification looking for abnormal cells. Second, the screener must cope with habituation (the expectation of a negative result) and fatigue. These limitations affect both the sensitivity and specificity of testing.

In addition to these technical aspects, conducting standard, manual CvC screening requires a sufficient quantity of qualified cytotechnologists and cytopathologists. This quantity will determine the volume of Pap smear slides that can reasonably be screened by such trained personnel. While the Clinical Laboratory Improvement Amendment of 1988 limits the number of slides that can be reviewed in an eight-hour day to 100, such a level of productivity is highly unlikely (Rosenthal D L, Automation and the endangered future of the Pap test, J Natl Cancer Inst 90:738-749, 1998). A more reasonable optimal number is 60 per 15 days, while an average would be 40-50. This translates into an individual cytotechnologist having the capacity to screen 10,000-14,000 slides per year. In the US there are approximately 4,800 certified cytotechnologists to screen the 50-60 million Pap smears done every year, which is an adequate number. However, many countries in the world lack a sufficient number of qualified personnel.

The development of LBC technology (ThinPrep, Cytyc Inc.; SurePath, TriPath Imaging, Inc.) has provided more standardized method of sampling, thus reducing by >50% the number of inadequate cell samples, while enabling the detection of 65% more LSIL in the general population than conventional Pap smear. In addition, automated slide preparation and automated reading methods have reduced the burden of the analysis, as well as the intra and inter-individual variability of evaluation. Nonetheless to date the Pap test, even in its LBC format, has limited sensitivity (50%), remains a screening based on subjective visual reading of cell morphologies, and requires appropriate infrastructure and human resources, making the tests labor-intensive and expensive.

It is noteworthy, that in spite of the annual screening of over 50 million women in the US where expertise and infrastructures are widely available, over 12,000 new cases of CvC were discovered in the year 2010 alone, with 3.5 millions referred to colposcopy examination, as a result of suspicious Pap smear results.

Alternatives to Pap smear. Colposcopy is the primary diagnostic method used in the US to detect CIN and cancer, following an abnormal Pap smear. Colposcopy is the visual examination of an illuminated and magnified view of the cervix and adjacent tissues, with the aim of distinguishing normal from abnormal tissue areas. First developed in 1925, this procedure is considered more accurate than Pap smear. A colposcope is a binocular microscope with built-in light source and objective lens attached to a support mechanism; it is positioned at about 30 centimeters from the vagina and allows magnification of the relevant area at low (2×, 6×), medium (88×, 15×) or high (15×, 25×) powers (Apgar B S, Brotzman G L and Spitzer M, Colposcopy: Principles and Practice, WB Saunders Company: Philadelphia, 2002).

A colposcopic examination involves a systematic visual evaluation of the lower genital tract (cervix, vulva and vagina) and is typically a painless procedure. The purpose of a colposcopic examination is to identify and rank the severity of lesions, so that abnormal areas may be biopsied if necessary. To this end, a 3-5% acetic acid solution is applied to the cervix, causing abnormal and metaplastic epithelia to turn white. Cervical cancer precursor lesions and invasive cancer exhibit certain distinctly abnormal morphologic features that can be identified by colposcopic examination (Coppelson M, Dalrymple J C, Atkinson K H, Colposcopic differentiation of abnormalities arising in the transformation zone, Contemp Colposcopy, 20:83-110, 1993; Reid R, Krums E P, Herschman R, et al., Genital warts and cervical cancer V. The tissue basis of colposcopic change, Am J Obstet Gynecol 149:293-30, 1984; Benedet J L, Anderson G H, Boyes D A, Colposcopic diagnosis of invasive and occult carcinoma of the cervix, Obstet Gynecol 65:557-562, 1985). Lesion characteristics such as margin shape, color or opacity, blood vessel caliber, intercapillary spacing and distribution, and contour are considered by physician colposcopists to reach a clinical diagnosis. The examination of these colposcopic signs determines the severity of the neoplasia and discriminates abnormal findings from similarly appearing, anatomically normal variants.

Most colposcopes are presently equipped with stereoscopic microscope head allowing a three-dimensional view through the binocular eye pieces, or with digital color camera and video packages, allowing for image storage and retrieval, and image display on large screen. A variety of light filters are also available to highlight different aspects of the cervix surface.

Colposcopy is the gold standard in the developed world for diagnosing cervical abnormalities after an abnormal Pap smear, yet it requires trained colposcopists and can be expensive to perform. Hence, other less expensive visualization techniques have been developed which can be performed with significantly less training, such as cervicography and speculoscopy.

Cervicography is a technique equivalent to colposcopy, for photographing part or all of the cervix. In fact, it consists of the use of a camera taking images of the cervix upon application of a 3-5% acetic acid solution. The photographs, referred to as cervigrams, are static images of the cervix similar to those seen during low-magnification colposcopy, and are sent to clinical laboratory for analysis. Equipped with a digital camera video terminal, digital cervicography further offers the merit of instantly confirming the quality of an image without having to call back the patient. Furthermore the digital feature allows for easy storage, retrieval, and screen viewing of the image with zoom features and the like. Cervicography is a diagnostic medical procedure in which a non-physician takes pictures of the cervix and submits them to a physician or central laboratory for interpretation by colposcopists. The procedure is still under investigation for three uses: i) as an alternative to Pap smear screening as a primary screening technique for cervical cancer, ii) as an adjunct to routine Pap smear to improve the sensitivity of Pap smear screening for cervical cancer, and iii) as a triage technique for colposcopy in patients found to have low-grade lesions on Pap smear specimens (Colposcopy, cervicography, speculoscopy and endoscopy, International Academy of Cytology Task Force summary. Diagnostic Cytology Towards the 21st Century: An International Expert Conference and Tutorial. Van Niekerk W A et al., Acta Cytol 42:33-49, 1998).

Speculoscopy refers to an endoscopic visual examination of the cervix that uses specialized blue-white chemiluminescence along with acetic acid solution and low-power magnification, similarly to colposcopy and cervicography. In contrast however, after application of the acetic acid solution, during speculoscopy, lights are dimmed and the cervix is visually examined using a magnifying lense and a disposable blue-white chemiluminescence light that is attached to the speculum blade. Epithelial cells with increased keratinization and nuclear cytoplasmic ratio have an increased light reflection and appear white, in clear distinction to the clear blue of the normal epithelium (Van Niekerk et al., 1998). The presence of white lesions is considered a positive result, prompting sampling for cytological evaluation. Proposed uses of speculoscopy are as an adjunct to conventional Pap screening and as a method of triage of women with atypical Pap smears prior to colposcopy. Note that cervicography and speculoscopy have not been fully validated in large clinical trials, and are still considered investigational techniques.

It is relevant to note that endoscopy methods have been in use for a long time. Endoscopy is a general term referring to the visual examination of an internal part of the human body or of a hollow organ, such as the gastro-intestinal tract (colonoscopy) or respiratory tract (bronchoscopy). Among gynecological applications, transvaginal endoscopy is used for the examination of the whole female reproductive tract as well as for small operative interventions of the genital apparatus. Endoscopic examinations are performed for diagnostic purposes or surgical procedures of the uterus and ovaries. The first endoscope was developed as early as the 1800s. Endoscopes have evolved, yet nowadays they still basically consist of: a) a rigid or flexible tube; b) a light delivering system to illuminate the object, the light being inside or outside and then directed via fiberoptics; c) a lens system transmitting the image from the objective lens to the viewer by a lens relay system or fiberoptics; d) an eyepiece; e) an additional channel within the tube to allow entry of medical instruments or manipulators.

Another low-cost visualization methodology is the “naked eye” visual inspection with acetic acid (VIA) or Lugol's iodine (VILI). VIA and VILI are being tested in low infrastructure settings, where Pap smear is not available. These simple procedures do not necessarily require doctors, or trained colposcopists or pathologists, yet can be effectively performed by a wide range of health professionals, including nurses, or midwives. Women presenting with abnormal VIA or VILI can be immediately identified and referred for further intervention. In studies, VIA has shown to perform as well as or better than cervical cytology in accurately identifying pre-cancerous lesions: mid-level providers were able to identify between 45% and 79% of women at high-risk of developing CvC (Sherris J et al. Evidence-based, alternative cervical cancer screening approaches in low-resource settings. International Perspectives on Sexual and Reproductive Health. 35(3), September 2009; Sankaranarayanan R. et al., A critical assessment of screening methods for cervical neoplasia, Int J Gyn Ob 89:Suppl 2:S4-S12, 2005).

Digital imaging display. With “naked eye” visual examination at one end of the methodology spectrum, digital imaging and image analysis algorithms are revolutionizing medical imaging and enabling sophisticated computer programs to assist the physicians with Computer-Aided-Diagnosis (CAD). Current algorithms tend to correlate clinical status with optical and physical properties of the patient cervix. In one study, the computer system was readily able to discriminate CIN 3 from normal epithelium and immature metaplasia (Dickman E D, Doll T J, Chiu C K, and Ferris D G, Identification of Cervical Neoplasia Using a Simulation of Human Vision, Journal of Lower Genital Tract Disease 5(3):144-152, 2001). Various image processing algorithms have been developed to detect different colposcopic features. Yang et al. developed a segmentation algorithm to detect acetowhite epithelium using a statistical optimization scheme (deterministic annealing) for accurate clustering to track the boundaries of the acetowhite regions (Yang S, Guo J, King P, Sriraja Y, Mitra S, Nutter B, Ferris D, Schiffman M, Jeronimo J, and Long R, A multi-spectral digital Cervigram™ analyzer in the wavelet domain for early detection of cervical cancer, Proceedings of SPIE on Medical Imaging, Vol. 5370 Bellingham, Wash. 2004, pp 1833-1844). Gordon et al. developed a segmentation algorithm for three tissue types in cervical imagery (original squamous, columnar, and acetowhite epithelium) based on color and texture information (Gordon S, Zimmerman G, and Greenspan H, Image segmentation of Uterine Cervix images and or video images for indexing in PACS, Proceedings of the 17.sup.th IEEE Symposium on Computer-Based Medical Systems (CBMS '04), 2004). Further, Balas (Costas Balas, A novel optical imaging method for the early detection, quantitative grading, and mapping of cancerous and precancerous lesions of cervix, IEEE Transactions on Biomedical Engineering, Vol. 48, No. 1, January 2001, 96-104; U.S. Pat. No. 7,598,088) and Orfanoudaki et al. (Orfanoudaki I M, Themelis G C, Sifakis S K, Fragouli D H, Panayiotides J G, Vazgiouraki E M, Koumantakis E E, A clinical study of optical biopsy of the uterine cervix using a multispectral imaging system, Gynecologic Oncology, Vol. 96, 119-131, 2005) analyzed the temporal decay of the acetic acid whitening effect by measuring the intensity profile over time. Furthermore, several approaches for tissue classification have been developed, such as a simple colposcopic image classification method by artificial neural network using the lesion contour features (Claude I, Winzenrieth R, Pouletaut P, and Boulanger J C, Contour Features for colposcopic image classification by artificial neural networks, in Proceedings of international conference on Pattern Recognition, 771-774, 2002), a rule based medical decision support system for detecting different stages of CvC based on the signs and symptoms from physical examination (Mitra P, Mitra S, and Pal S K, Staging of Cervical Cancer with Soft Computing, IEEE Transactions on Biomedical Engineering, Vol. 47, No. 7, July 2000, pp 934-940). U.S. Pat. No. 7,664,300 reports the development of a CAD system for uterine cervical cancer whereby an algorithm analyzes data acquired from the cervix such as color or video images, reflectance and fluorescence multi-spectral or hyper-spectral imagery, impedance etc. The CAD system may eventually establish diagnosis and is proposed with utility application as adjunct to colposcopy. Teachings from that patent focus on the development of an image analysis and recognition system correlating cervical images, based on measurable cervical parameters, to a cervical clinical status.

In conclusion, while several methodologies have been separately developed for cervical examination and screening, none of the above mentioned technologies encompass the present invention, which comprises: introduction of a digital imaging device in the woman's body, image capture of the cervix and its environment, self-operability, on-site as well remote analysis of patient cervical images, and image analysis and comparison to a remote central reference database, as further described in detail below.

There is still a great need to overcome the limitations of subjective Pap smear reading, and avoid missing dozens of thousands of CvC patients. Indeed, the latter must have been consecutively misdiagnosed, year after year, in spite of their annual attendance to cervical screening. There is also an even greater need to provide to millions of women in many countries of the world a way to access regular screening as well as professional expertise, regardless of how remote they might be from health care centers. The present invention offers a solution to these needs.

### SUMMARY OF THE INVENTION

Pap smear has undoubtedly contributed to the steady decrease in incidence and mortality rates from cervical cancer in developed countries. However Pap test has insufficient sensitivity, recently estimated to only 51%, suffers from high susceptibility to intra and inter-individual reading variability, and requires highly trained staff and adequate laboratories, making the tests labor-intensive and expensive. There is a need for other methods that may be used as adjunct to the Pap test to increase its accuracy. There is also a need to provide increased access to cervical screening to women, particularly in less urban areas and underserved countries, including access to highly trained experts, and to improve cervical disease surveillance.

The invention relates to providing a user-friendly digital cervical examination and evaluation system that can be either self-operated in the privacy of one's home, or practiced in the doctor's office in view of cervical disease surveillance, management and screening.

The digital device contemplated in this invention may take images and/or video images of the cervix and vaginal cavity, store them, and display them. Images can then be either observed and examined on-site, or transferred to a health center, even distantly located, for further examination and analysis by health professionals.

This device facilitates cervical screening by improving cost-effectiveness and frequency of examination. This system also enables a patient to create her own personal medical history through an image library that may be instrumental in early detection of disease.

The device and system of the present invention will be particularly valuable in remote parts of the world where infrastructure is not well developed and health professionals are rare. This device will thus allow every woman to gain access to high standards of health care and to health professional expertise, with respect to cervical examination and cervical disease screening.

The device of the present invention is composed of: a flexible tube for insertion into a human body cavity, a digital camera and a light source, both located at the internal end of the tube, the flexible tube carrying a bundle of fiberoptics system connecting the digital camera to an outer digital mean, including but not limited to a computer system, through a USB-type connection. The external end of the tube also carries the command controls necessary to activate the light and camera.

The device of the present invention is thus designed to be inserted into a woman's vagina, and to collect images and or video images of the cervix and its environment, which are displayed, stored and analyzed, via a computer-based system either on-site or at a remote health center, by experts in the field of cervical cancer and gynecology. Communication devices such as cell phone, smart phone, IPhone and desk top computers are contemplated as means to send the collected images and or video images for expert analysis to the health care center.

The present invention also encompasses the use of a memory card associated to the digital camera cervical examination, a method that further emphasizes the self-operability of the device of the present invention, and further facilitates the transfer of patient's images to a health center for analysis, even in remote areas.

The device of the present invention provides a better alternative to colposcopy and an adjunct to Pap smear. Indeed, the device of the present invention provides a user-friendly system that can be easily operated by a patient in the comfort and privacy of her home, in view of cervical disease screening, surveillance or treatment follow-up. Insofar, the device of the present invention can be used for the scope of a frequent home-based cervical examination in addition to a yearly Pap test.

The present invention encompasses the use and application of the device and system described herein to other endoscopic procedures, including but not limited to bronchoscopy, colonoscopy, and sigmoidoscopy.

In conclusion, the device of the present invention has utility application in the surveillance, detection, screening and disease management of cervical disease both in developed and developing countries.

The invention further provides a system enabling health experts to compare patient images to a reference database of cervical lesions of different grades, due to dysplasia and cancer, as well as to images of other cervical conditions such as inflammation of the cervix and vaginal cavity, and infection of these organs by different pathogens. The use of such reference database has utility application in providing and validating diagnosis.

### BRIEF DESCRIPTION OF THE DRAWINGS

FIG. 1: Schematic diagram of the digital device and process of the present invention. A. Schematic illustration of the digital device that is used for cervix and vaginal cavity examination. The device consists of a flexible tubing with an “internal” and a “distal” end. The internal end carries a digital camera and light source, while the distal end carries a USB-type connection or portal as well as an electric connection for the light source. The device can be either used by a health care professional on a patient, or can be used by the patient herself via a self-examination procedure, as shown in FIG. 6 below. B. The images taken of the patient cervix by the device can be displayed and viewed, via the USB-type connection, through a computer, or a cell phone, or any equivalent digital mean. The images can thus be immediately analyzed by a health care professional, or can be stored, shared and transferred to a remote health center site (C) connected to a remote central database of cervical lesions (D). The use of a memory card as a means to store the images and transfer them to the health center is encompassed herein. C. The third step in this process is the analysis of the images or video images by health care professionals in the health care center (C), and their comparison to a central reference database of cervical images (D), in view of establishing a clinical decision and diagnosis.

FIG. 2: Images of a normal cervix. The figure provides a longitudinal section of the female genital apparatus, indicating the position of the vagina, cervix and uterus. A normal cervix appears as a doughnut shaped organ with a central opening, and is characterized by a pink color and a smooth surface of the tissues.

FIG. 3: Cervical cancer development. The figure schematically illustrates a cervix at different stages of cervical cancer development: normal, low-grade cervical intraepithelial neoplasia (CIN), high-grade CIN, and cancer.

FIG. 4: Images of cervix with cervical cancer. These various images illustrate that an abnormal cervix affected with cervical cancer exhibits a very distinct appearance with respect to a normal cervix (see FIG. 2). The surface is not smooth, and the tumor growth is distinct and obvious from the remaining normal portion of the organ. These features would be easily recognizable even by non-health professionals.

FIG. 5: Images of abnormal cervix. The various images below illustrate abnormal cervix affected with different conditions: inflammation (A), herpes virus infection (B), and Chlamydia infection (C). Note the typical pink color of a normal cervix on the left side, compared to the red color appearance of the cervix in the case of cervicitis (A).

FIG. 6: Uses of the system of the present invention. A. Use by a health care professional. Schematic representation of the sagittal section of a woman genital apparatus, including a vaginal speculum introduced for the purpose of cervical examination. The figure also illustrates a woman being examined in the doctor's office, positioned for pelvic examination. Note that the health care professional may be able to use the device of the present invention, capture, display and analyze the images on-site, or transfer them through the portal to a health care center with access to a reference database of cervix images. B. Self-use. A woman may in the comfort and privacy of her own home use the device by herself or with the assistance of a selected trusted individual. The images thus taken can be viewed immediately through a computer or even a cell phone through the portal connection. The latter also enables to electronically send and transfer the images to the health center for analysis.

FIG. 7: Process of cervical examination with digital imaging device and data transfer to remote digital reference laboratory through patient specific memory card. Cervical self-examination enables the patient to collect images through a cell phone for example. The memory card carries a bar code identifying the patient, and is attached to the cell phone or the digital camera device itself. The memory card is removed and mailed to a reference laboratory for analysis. The reference laboratory, thus collecting thousands of patient images, builds its own reference library of images for improved analysis and diagnostic power. The memory card is then sent back to the patient or to the doctor's office together with the diagnostic report.

### DESCRIPTION OF PREFERRED EMBODIMENT

The standard method of CvC screening uses the Pap smear which is presently a two-step process involving: a) sample collection from the cervix and b) upon cytological staining, visual examination of the sample under the microscope for the potential presence of abnormal cells. There are inherent limitations in this standard screening approach, which ultimately affect overall results and sample diagnosis. Major limitations, known to those skilled in the art, are related to the quality of the sample, the quality of the slide, the effectiveness of the screener, the representative examination of all cervical cell types on the slide, the inadequate layering of the sample. These limitations are routinely observed even in countries with adequate infrastructure and expertise.

Furthermore, despite recommended annual cervical screening and the fact that CvC is a slow disease that may take many years to develop, dozens of thousands of women in the US alone are discovered every year with CvC. This suggests that the newly diagnosed women with CvC must have been missed several times during Pap smear screening to eventually develop cancer. It also demonstrates the limited sensitivity of the standard Pap smear screening procedure.

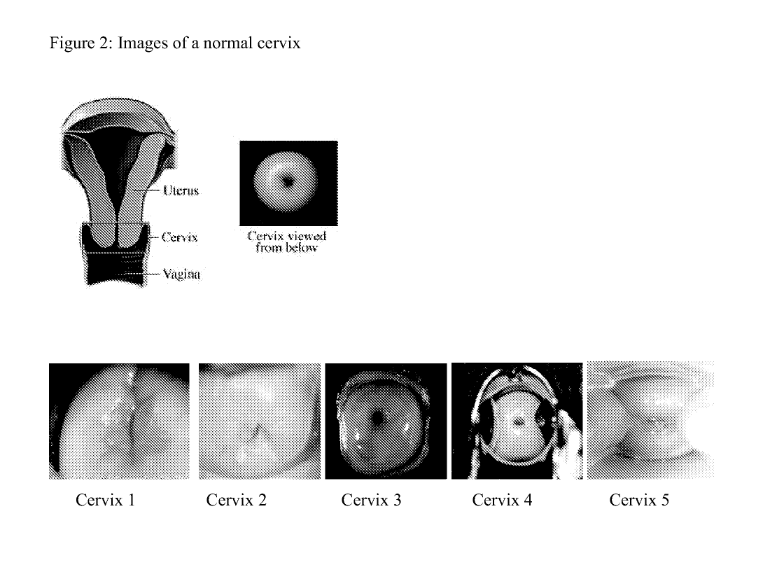

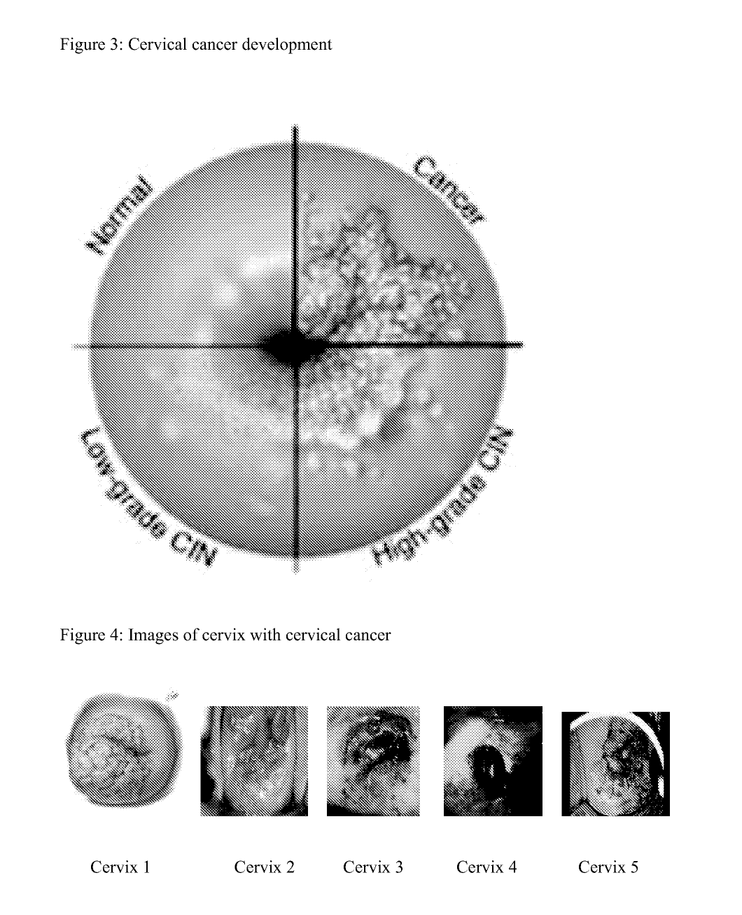

It is important to note that when Pap smear results are suspicious, the patient is referred to colposcopy, a visual examination of the cervix under magnification. The colposcopist will observe the basic characteristics of the cervix, such as its surface (smooth, rugged, with extra tissue growing), texture, color, appearance, morphological changes, and vascularization (FIGS. 2-5). It is only when the examiner observes suspicious features of the patient cervix through direct visual examination that biopsy collection and histological examination are considered. Therefore it is the visual examination of the cervix upon colposcopy that ultimately provides the most accurate diagnosis as compared to routine Pap smear screening.

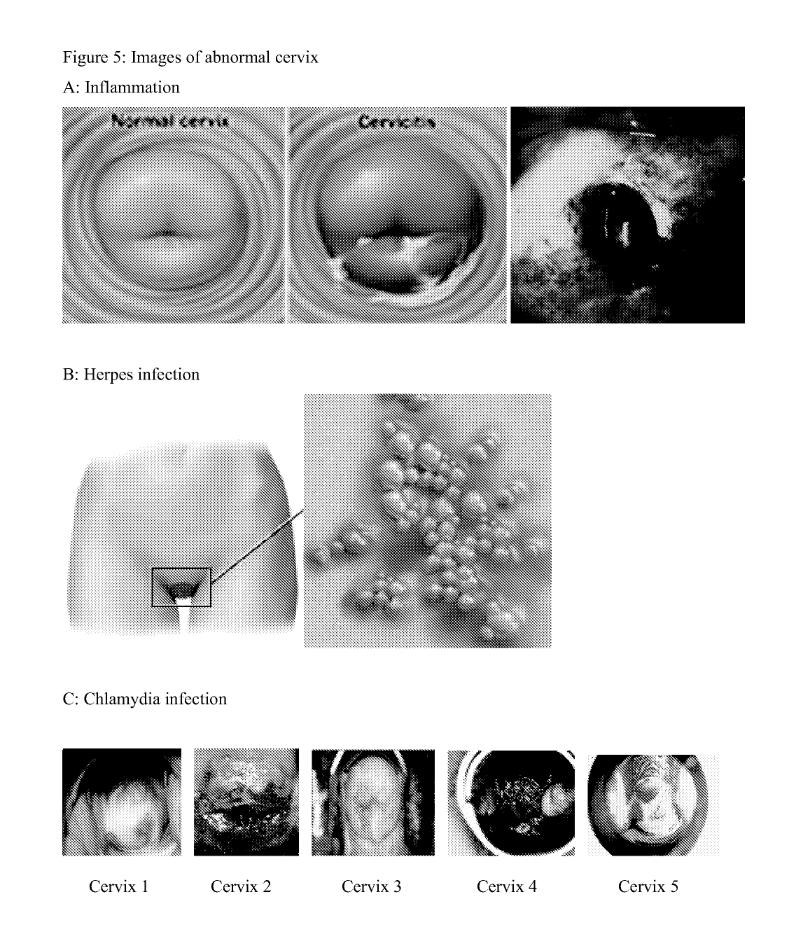

Not only colposcopy is instrumental to CvC diagnosis, but also to other conditions such as polyps, cervical inflammation or infection with herpes, Chlamydia and other infectious diseases (FIG. 5). Colposcopy however still requires an adequate infrastructure setting, and a health professional. Insofar, such procedure may not be applicable as a routine screening in developed countries, and much less in developing countries.

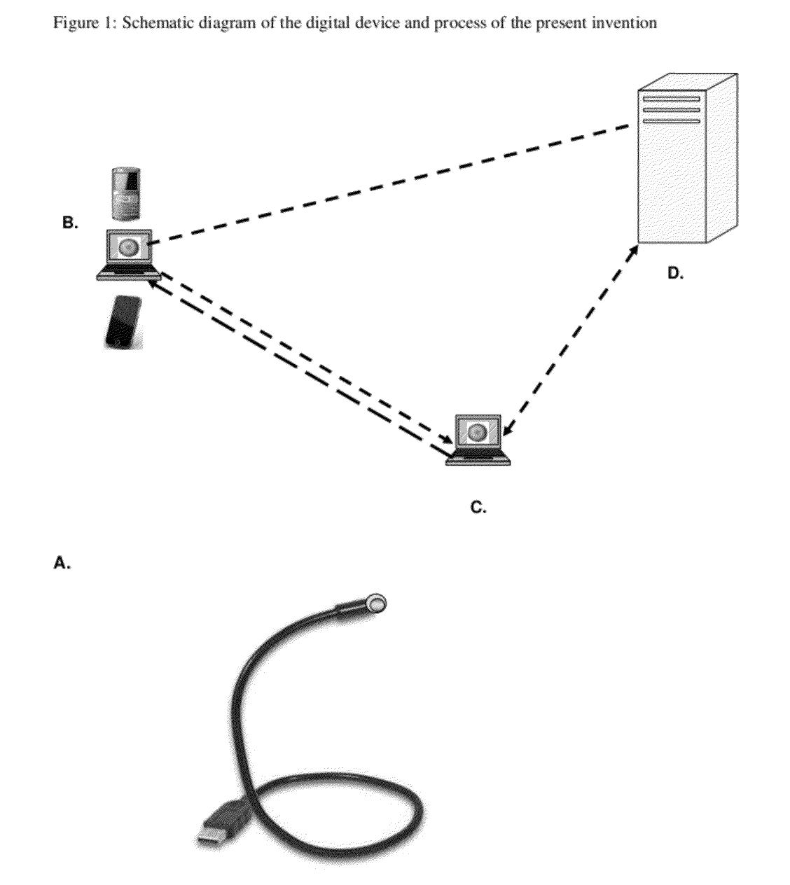

Description of the device and system. The invention relates to providing a user-friendly digital cervical examination and evaluation system that can be either practiced in the doctor's office in view of cervical disease surveillance, management and screening, or self-operated in the privacy of one's home. The easy-to-use device of the present invention is capable of taking images and video images of the cervix and vaginal cavity, storing, displaying and transferring them via digital means (FIG. 1).

The device of the present invention is composed of: a flexible tube for insertion into the human body, a digital camera and a light source, both located at the internal or proximal end of the tube, the flexible tube carrying a bundle of fiberoptics system connecting the digital camera to an outer distal digital mean with the capacity to collect, store, display captured images or video images, and transfer them as required. The external or distal end of the tube also carries the command controls necessary to activate the light and camera. The digital camera in this device is equipped with low and high magnification capability. High quality miniature digital camera are commercially available, and come in different shapes, including but not limited to pen-like objects.

The flexible tube of the device of the present invention is of approximately one centimeter in diameter or preferably less, and is preferably flexible enough, to allow its manipulation in such a way that it will remain at a certain angle degree if required. This flexibility will allow for better visualization of the cervix environment. This flexible tube may contain accessory cables of smaller dimension that possess different connections at both ends. Although the tube is preferably flexible, other types of tubing, semi-rigid or rigid, are encompassed by the present invention, including but not limited to connectors similar to tampon applicators. Introduction of the device of the present invention into the vaginal cavity may be preferably accomplished after the introduction of a vaginal speculum, the use of which is encompassed herein. A vaginal speculum is commonly introduced in the vagina in view of cervical examination and cervical specimen collection.

Within the flexible or connecting tube, there are two cables, referred to herein as the “light cable” and “the digital cable”. The light cable will transmit light from a light source located at the distal end of the connecting tube, to the proximal end to shed light on the object from which images and video images are to be taken by the small digital camera. Light could be carried through fiberoptics or any other mean with the capability of transmitting light. The digital cable will connect the proximal end small digital camera to a USB-type connection located at the distal end of the digital cable; said USB-type connection is adaptable to an external digital display mean. The digital mean referred to herein is preferably a computer, although other electronic devices are encompassed by the present invention, including but not limited to, a cell phone, an iPhone, a smartphone, or any other similar electronic digital mean with the capacity to store, display, share, transfer and send the collected images or video images to a remote site. Both internet and wireless connection are encompassed by the present invention. The connection of the device via its USB portal to the digital mean will allow the display of the images and video images taken by the digital camera.

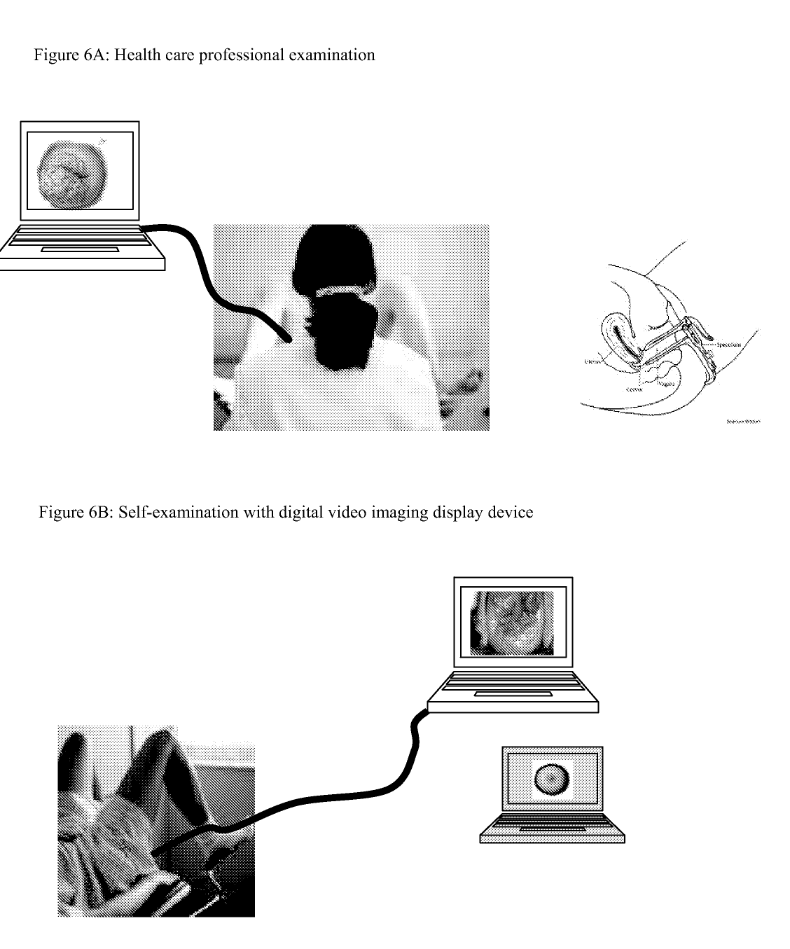

This instrument can either be used by a health practitioner in the doctor's office, or self-operated by the patient herself (FIGS. 6A and 6B). Images may be examined and analyzed by health professionals either on-site or at a remote health center, while patient may observe images of her own cervix prior to transfer them to the health center.

The device and system of the present invention includes a software application that can be loaded on a digital means such as desktop computer, smart phone, iPad, or other equivalent digital means. This application will permit the control and operation of the device, including the light and the digital camera, and allow image and video image capture at the desired magnification, as well as image display, sharing and transfer. The system will thus display color, texture, and morphology of the patient cervix, and vaginal cavity. Furthermore, the software application may also allow comparison of a patient cervix images to a remote central database of thousands of images of cervical disease and cervical lesions, including cancer at different stages of development, inflammation, and pathogen infection, etc. (FIGS. 2-4). Analysis of the modifications and differences between the patient images and those of the reference database, allows the software to even provide patient diagnosis upon comparison of the patient cervix images to those in the reference database.

Hence, the system of the present invention preferably comprises a software for digital camera operation and a software for image analysis and recognition. They are preferably combined, or alternatively they are separate entities and can even be added on at a later time.

It is important to note, that at present there are already automated screening machines for the analysis of Pap stained slides using automated processing, such as the FDA approved AutoPap 300 CQ (Tri-Path Imaging, Inc.) which removes 25% of all samples from manual review, and the AutoPap GS and AutoCyte SCREEN. Similarly, there are FDA-cleared digital imaging analysis systems that are commercially available and allow pathologists to run quantitative immunohistochemistry image analysis while reading slides on a computer monitor rather than through a microscope eyepiece.

The concept of transmission of digital images to a remote health center for further analysis, remote image reading and reporting has been described in U.S. Pat. No. 6,542,456. As a solution to the lack of cervical cancer screening in low-infrastructure countries, U.S. Pat. No. 6,542,456 teaches how the first steps of Pap smear screening, including sampling, slide preparation and automated first reading, as performed at a first geographic location, may serve to select potentially positive slides, whose images are then electronically sent to a second remote geographic location, preferably located in a developed country, where experts read the suspicious slides and electronically report their findings back to the first geographic location.

The system of the present invention includes use of acetic acid as a signal enhancing contrast agent to allow better discrimination of normal and abnormal cervical tissues, as practiced in the colposcopy method.

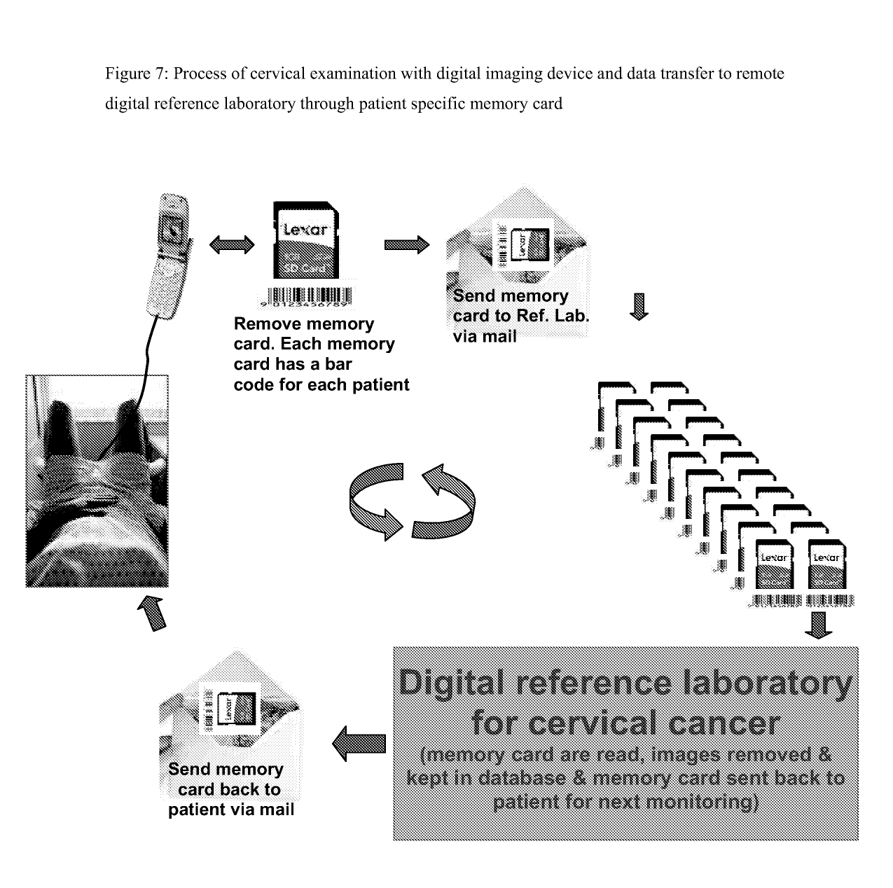

In a preferred embodiment of the present invention, the device and system comprise a memory card. Said memory card can be purchased for the scope of cervical examination, either by an individual or by a doctor's office, said memory card being identified by a bar code associated to the patient and doctor's office. In the absence of a computer system or cell phone or other electronic device for electronically transferring cervical images to the health center, the memory card can be mailed to the health center responsible for the analysis. The card can be mailed back with the results to the patient or doctor's office (FIG. 7).

In another embodiment of the present invention the device and system of the present invention can be extended and applied to other endoscopic procedures, including but not limited to bronchoscopy, colonoscopy, and sigmoidoscopy. Indeed, digital display, storage, and transmission of the images can be successfully provided by the present invention to other endoscopy procedure. In fact all the features of the present invention, that have been described in detail herein, can be applied to and benefit any other endoscopy procedures, including but not limited to: on-site and remote analysis, access to health professionals and specialists even to patients living in underserved areas, generation of a patient's personal medical record, comprising images taken at different organ sites and over time, increased cost-effectiveness of the procedure, increased frequency of screening, user-friendly transfer of personal medical data, etc. This invention applies with particular emphasis to sigmoidoscopy that unlike colonoscopy does not require sedation, yet only visualizes the lower third of the colon. In the case of sigmoidoscopy, even self-operation with the device and system of the present invention may be envisaged by providing appropriate instructions to the patient, and minor modifications to the device as known to those skilled in the arts.

Educational material and instructions for use. The digital imaging device of the present invention is provided with well-designed educational material and easy-to-follow step-by-step instructions for use, offering clear and self-explanatory schematic diagrams. The level of understanding associated with the use of the device of the present invention appears analogous to the level of understanding required for the proper use and insertion of tampons, or contraceptive devices such as but not limited to, diaphragms, cervical caps, female condoms, or interpretation on the use of ovulation tests, pregnancy tests, and the like. The educational material included with the device of the present invention makes the use of the digital imaging device very simple. In addition, no further knowledge than the one involved in the use and manipulation of a commonly used home-based computer, or cell phone, or of any touch-based electronic device is required to operate the digital device and system of the present invention.

The use of the device of the present invention requires an “operator”, the one introducing the device in the patient body, and performing the image capture and collection. The operator may be either a health care professional, although not necessarily a gynecology doctor (see below: use by a health care professional, and FIG. 6A), or the patient herself (see below self use, and FIG. 6B). In the self-mode use of the device, the operator may also be an age-appropriate trusted lay person selected by the woman who wishes to have the images taken for the purpose of cervical disease detection and screening, including but not limited to a household or family member, such as a spouse, friend, companion, or partner. In the self-mode use of the device, it is understood that the operator preferably has at least elementary education to safely and properly accomplish the simple procedure of the present invention while referring to the educational material and instructions for use.

The instructions for use of the device of the present invention are most important when the device is operated in the “self-mode”. The instructions will preferably include how the woman should best prepare for using the device, including recommendations such as but not limited to performing the examination outside of her menstrual period, not using douche or taking a bath, or using contraceptive creams for 24 hours before the test, or not having sexual activity for 24 hours before the test. These recommendations are typically made by the doctor's office when a woman is scheduling a Pap test. In the context of the present invention, such recommendations will be provided in the attached instructions for use.

The instructions for use preferably describe and illustrate step-by-step the adequate insertion of the digital imaging device through the vagina. In particular, the use of a sterile disposable vaginal speculum in conjunction with the digital imaging device is encompassed by the present invention, and described in detail in the instructions for use. The instructions for use will also preferably include recommendations on how to successfully take representative images and video images with the device, in terms of magnification and positioning of the camera at an appropriate distance from the cervix itself. In addition, they will preferably recommend the viewing of the images on a digital display mean, prior to their storage and transfer to the health center to ensure the optimal quality of the images. Finally, the instructions for use of the present invention will also preferably include recommendations on how to successfully use the device in the most comfortable manner as possible, and create environmental conditions that ensure operator ease and relax the patient.

Use of the digital device by health professional. In a preferred embodiment of the present invention, the device may be used in a doctor's office, in a clinical laboratory, in a hospital or any other similar setting. In this case the health professional will position the patient as for a pelvic examination or Pap smear screening (FIG. 6A). Upon introducing a vaginal speculum as for cervical examination, the health practitioner will preferably use a solution of 3% acetic acid to better discriminate normal from abnormal areas of the cervix, as commonly practiced during colposcopy, and will use the device to capture images and video images of the patient cervix (FIG. 6A). The images may be displayed for immediate observation using a digital display such as a computer and may be even shared with the patient on-site. The patient may therefore not only see real time pictures of her own cervix, but may also interact with the examiner and ask questions, and clarifications. The images may also be transferred to a health center for analysis and comparison to a reference database, and clinical diagnosis is returned to the doctor.

The device and procedure of the present invention are discomfort free and offer a quality of examination at least comparable to that of colposcopy. If cervix images appear normal, patient is released and asked to return for future tests. On the contrary, abnormal cervix images may immediately prompt on-site biopsy when the procedure is performed in the doctor's office.

The device of the present invention has improved clinical utility and application with respect to current cervicography and colposcopy, because it combines colposcopy level of evaluation with the added benefit of self-mode use and the remote analysis and comparison to reference database.

Self-use of the device and system of the present invention. As described in the instructions for use accompanying the device and system of the present invention, the patient will position herself similarly to a pelvic examination in a doctor's office (FIG. 6B), preferably comfortably lying down on her back on a bed, or couch with feet on a coffee table. First, the patient will introduce a sterile disposable speculum into the vagina, and set it into an adequate opening, according to the instructions for use. Second, the patient will preferably use a cotton tipped applicator or swab, of approximately 10 to 12 inches long, dipped in a sterile 3% acetic acid solution, that she will gently introduce into the speculum opening, and she will apply said solution over the surface of her cervix. Within two to three minutes, the acetic acid reaction takes place, and abnormal areas turn to white. Third, the patient will preferably connect the USB-type distal end of the device of the present invention to a digital display mean, such as desk computer and position the screen of the digital display in order to view the images in real time, as they are taken. Finally, the patient will gently introduce the proximal end of the device (the one with a digital camera and a light) into the vagina, and operate the digital display to view the cervix and its environment. The patient on her own or with the assistance of a trusted “operator” may open the software application in the digital device and control the operations: image capture, view, storage, and transfer to a remote health care center location for further examination, analysis and diagnosis.

Clearly, self-use of the device and system of the present invention offers several advantages: 1) ease of operation enabling the patient to perform cervical examination in the privacy and comfort of her home, and as often as desired, to gain real time information on her cervix and its environment, 2) possibility for any patient to capture relevant images, store them in a personal database for future reference, to document and monitor relevant changes of these organs over time, 3) possibility to send the images and video images to a remote health center where experts may examine said images, and provide diagnosis that is communicated back to the patient and/or her practitioner, and finally 4) providing at least the same quality of analysis as obtained upon colposcopy, as well as “colposcopy quality” diagnosis remotely without the inconvenience and discomfort of the real colposcopy examination in the doctor's office.

Taken together these advantages make cervical disease screening widely accessible and affordable to any woman, particularly those living in remote areas, and those who do not have access to high quality health care infrastructure.

The device and system of the present invention will also contribute to more frequent screening leading to improved surveillance and disease management, increasing the chance of identifying early lesions that are more amenable to treatment, as well as monitoring women who are at higher risk for sexually transmitted diseases and cervical disease. As further discussed below, although, many countries lack medical infrastructure and/or technical expertise to carry out effective mass screening via conventional Pap smear, the use of the present invention will result and translate into a decrease in the incidence and mortality of CvC in women in the developing world.

Utility application in developed countries. In developed countries, despite routine Pap screening and CvC slow progression, thousands of women are still been diagnosed every year with CvC. This suggests that the newly diagnosed women with CvC must have been consecutively missed during Pap smear screening to eventually develop cancer. It also demonstrates the limited sensitivity of the standard Pap smear screening procedure.

In developed countries, the digital device and system of the present invention will provide a better alternative to colposcopy and an adjunct to Pap smear screening. The system of the present invention offers a novel, cost-effective and user-friendly examination methodology. Hence the system enables more frequent examinations in view of cervical disease screening, surveillance or treatment follow-up, whether at the practitioner's office or as monitored in the comfort and privacy of one's home. Insofar, the device of the present invention can be used for the scope of frequent cervical examination surveillance in addition to a yearly Pap test. The capability of the system of the present invention to compare captured images to a reference database, offers additional benefit in terms of expertise of analysis, and reduction of false negative and false positive.

In developed countries, women are well educated in women's health care issues. Women in developed countries possess the skill and understanding to use the digital device of the present invention, and transfer captured images on a digital display mean such as a computer. They may also be able to make a reasonable judgment on the need for further examination by a specialist, based on the basic characteristics of the displayed images taken of their cervix, and based on the educational material and instructions for use. They may themselves interrogate the reference database to help interpretation. In all cases, they may elect to send the images to the health center via a digital mean such as internet for further examination, and receive in turn the diagnosis.

User-friendly instructions and schematic diagrams, the comfort and privacy of one's home, performing the test at one's convenience, eliminating the burden of scheduling a doctor's visit, and making time for the appointment are major features offered by the device and system of the present invention. Altogether, these features will be considered very beneficial by today's women in developed countries.

Thus, it is anticipated that the self-mode use of the device and system of the present invention will take rapid expansion in developed countries. Furthermore, by eliminating the need for a doctor's visit, it will eventually contribute to a decrease in health care costs.

In conclusion, it is contemplated that in developed countries, the present invention, whether through the health practitioner, or the self-mode use, by enabling cost-effective and frequent screening, will contribute to more regular surveillance of cervical disease incidence, in turn leading to a reduction of CvC incidence.

Utility application in low-infrastructure countries. As discussed above, the incidence of CvC is very high in the developing world as well as in low-infrastructure countries, where there is no structured healthcare, and the conventional Pap smear screening is either lacking or has limited coverage (ACS, 2007; Gakidou E, Nordhagen S, Obermeyer Z. Coverage of cervical cancer screening in 57 countries: low average levels and large inequalities. PLoS Med 5(6): e132. doi:10.1371/journal.pmed.0050132, 2008). Health care access and infrastructure, cost, unsafe sex, HPV infection burden, socio-economic status and education level, are all major reasons for the high morbidity and lack of screening. In addition, there are other factors that negatively impact on CvC incidence in women worldwide. Cultural, religious, and psychological factors are real barriers that exacerbate the problem of CvC screening.

The case of Mexico is particularly compelling. Despite a national CvC screening program that has been officially in place since 1974, CvC incidence is as high as 40.5 per 100,000 women, with a mortality rate of 17.1, as reported for the year 2000 (Arrossi S, Sankaranarayanan R, Parkin D M. Incidence and mortality of cervical cancer in Latin America, Salud Publica Mex 45 suppl 3:S306-S314, 2003; Lazcano-Ponce E, Moss S, Alonso De Ruiz P, Salmeron Castro J, Hernandez Avila M. Cervical cancer screening in developing countries. Why is it ineffective? The case of Mexico, Archives of Medical Research 30:240-250, 1999). As a reference, Bolivia and Haiti have an incidence rate of 58 and 94 (per 100,000), and a mortality rate of 22 and 53, respectively. In contrast, Puerto Rico boasts an incidence rate of 10 and a mortality rate of 4. A recent study (Leyva M, Byrd T, Tartwater P. Attitudes towards cervical cancer screening: A study of beliefs among women in Mexico, CA J Health Promotion, 4:13-24, 2006), performed among working women in a city of over one million people, exemplifies how beliefs enrooted in the population may adversely affect the overall participation of women even to a nationally sponsored program. Embarrassment of being examined, particularly by a male practitioner, embarrassment regarding sexual activity in general, misconceptions regarding cost and coverage, and where to go, believing the test being long or painful, lack of prevention awareness, fear of test results, misperceptions about the severity of the disease, all are elements that have been noted (Leyva, 2006).

In conclusion, there are two major issues affecting CvC screening in developing countries: access to health care and infrastructure on the one hand, and social and psychological factors on the other hand. The digital device and system of the present invention may contribute to overcome both.

Indeed the self-mode use of the device and system of the invention, and its instructions for use, enables the woman to have the test performed in the comfort and privacy of her home, eliminating fear, embarrassment and other psychological issues related to the doctor's visit. Furthermore, the relaxing and comfortable environment contributes to the quality of the experience, eliminating misconceptions about test length or pain. It is anticipated that facilitating access to the test, and rendering the test more patient-friendly, may also in turn promote more frequent testing.

On the other hand, the device and system of the present invention, allows every woman, regardless of how remote she can be from medical infrastructure and health professionals, to gain access to a high standard of health care with respect to cervical examination and cervical disease screening.

Conclusion. There is a need to increase accuracy and sensitivity of current cervical screening in developed countries, and to improve access to cervical screening in low-infrastructure areas and in developing countries.

The present invention reconciles the needs recognized above, and offers a system integrating several advantages simultaneously: a) low-cost hand-held device; b) with capability of capturing, storing, displaying, and ultimately sharing patient cervical images within database or among cytopathologists; c) facilitating access to health care professionals, even for women in rural or underserved areas; d) bypassing technical limitations of Pap smear as described above (sampling, intra and inter-variability, sensitivity); e) improving frequency and cost-effectiveness of cervical disease surveillance.

Altogether these features may ultimately lead to higher standards of cervical screening and disease management and to a decrease of false-positive and false negative rates of cervical screening.

The present invention could have clinical application and uses at least in the following areas: i) as an alternative to Pap smear screening as a primary screening technique for cervical cancer, ii) as an adjunct to routine Pap smear to improve the sensitivity of Pap smear screening for cervical cancer, and iii) as a triage technique for colposcopy in patients found to have low-grade lesions on Pap smear specimens.

While an embodiment of the present invention as been shown and described, various modifications may be made without departing from the scope of the present invention, and all such modifications and equivalents are intended to be covered.

What is claimed is:

1. A digital system for disease detection, screening and management, designed for insertion into a human body cavity, comprising a digital camera, memory card, and light source at one end and a digital portal connection at the other end, enabling capture, storage, display, transfer of images of the patient body cavity, both ends connected through a flexible, semi-rigid or rigid tubing containing a digital cable and a light cable, the former connecting the digital camera at one end to a digital display device at the other end, and the latter transmitting power to the light source at the internal end, said device and system being health practitioner-operated or self-operated, and enabling on-site and remote image analysis and evaluation.

2. The digital imaging device of claim 1 where the body cavity is a woman's genital apparatus and where the system is used to evaluate cervical lesions or abnormalities related to cancer, inflammation, infectious diseases, or other conditions.

3. The digital imaging device of claim 2 where the system is used in cervical disease diagnosis and management as adjunct to Pap screening.

4. The digital imaging device of claim 1 where the body cavity is the gastrointestinal (GI) tract and the system is used in GI explorations, GI disease detection, diagnosis, management and screening.

5. The digital imaging device of claim 1 where the distal end enables camera connection to a digital display and transfer device such as a desk top computer, cell phone or other electronic device designed to display images or video images and transfer them remotely.

6. The digital imaging device of claim 5 where data transfer involves collecting images on a memory card and transferring the memory card to a remote reference laboratory.

7. The digital imaging device of claim 1 where captured images are analyzed by remotely located health professionals and compared to archived images in a central reference database.

8. The digital imaging device of claim 2 where the patient cervix is wiped with acetic acid or equivalent solution in order to enhance visualization of abnormal cervical areas.

9. The digital imaging device of claim 2 where evaluation of patient cervical images relies on at least one of the following criteria, namely morphology, texture, color, surface appearance, including lesion presence, and vascularization.

10. The digital imaging system of claim 1 comprising software applications for digital camera operation, as well as for image analysis and recognition, the latter based on comparison of patient body cavity images to a reference database of archived images, each software application, being either included, alone or in combination, or separately added to the device.

11. The method of conducting a self-examination with the digital imaging device of claim 1, and the instructions for use, comprising introducing the device, connecting the device to a downstream digital mean, capturing body cavity images, displaying them on the digital mean, and transferring them, including by using a memory card, to a remote location, where they will be evaluated by health professionals in view of disease detection and screening, with respect to a reference database of archived images.

12. The method of claim 11 where the examination is conducted by a health practitioner and image evaluation is performed on-site or remotely, including by transferring images using a memory card.

13. The method of claim 11 and 12 where the body cavity is a woman's genital apparatus and the method includes swabbing the relevant area with acetic acid or analogous solution to enhance the visualization of abnormal areas.

14. The method of claim 11 and 12 where the body cavity is the GI tract.
